SEQUENCE LISTING (27 sequences)

SEQ ID NO: 1 - DNA, 285 bp, Pseudoplectania nigrella
  CDS (1)..(285)
  sig_peptide (1)..(69)
  mat_peptide (166)..(285)
atgcaatttaccaccatcctctccatcggtatcaccgtcttcggacttctcaacaccggagcctttgcagcaccccagcctgttcccgaggcttacgctgtttctgatcccgaggctcatcctgacgattttgctggtatggatgcgaaccaacttcagaaacgtggatttggatgcaatggtccttgggatgaggatgatatgcagtgccacaatcactgcaagtctattaagggttacaagggaggttattgtgctaaggggggctttgtttgcaagtgttac

SEQ ID NO: 2 - protein, 95 aa, Pseudoplectania nigrella
MQFTTILSIGITVFGLLNTGAFAAPQPVPEAYAVSDPEAHPDDFAGMDANQLQKRGFGCNGPWDEDDMQCHNHCKSIKGYKGGYCAKGGFVCKCY

SEQ ID NO: 3 - protein, 40 aa, Artificial sequence
  Synthetic construct
GFGCNGPWDEDDMKCHNHCKSIKGYRGGYCAKGGFVCKCY

SEQ ID NO: 4 - protein, 40 aa, Artificial sequence
  Synthetic construct
GFGCNGPWDEDDMQCHNHCKSIKGYRGGYCAKGGFVCKCY

SEQ ID NO: 5 - protein, 40 aa, Artificial sequence
  Synthetic construct
GFGCNGPWDEDDMFCHNHCKSIKGYKGGYCAKGGFVCKCY

SEQ ID NO: 6 - protein, 40 aa, Artificial sequence
  Synthetic construct
GFGCNGPWDEDDMRCHNHCKSIKGYRGGYCAKGGFVCRCY

SEQ ID NO: 7 - protein, 40 aa, Artificial sequence
  Synthetic construct
GFGCNGPWDEDDMRCHNHCRSIKGYKGGYCAKGGFVCKCY

SEQ ID NO: 8 - protein, 40 aa, Artificial sequence
  Synthetic construct
GFGCNGPWDEDDMLCHNHCKSIKGYKGGYCAKGGFVCKCY

SEQ ID NO: 9 - protein, 40 aa, Artificial sequence
  Synthetic construct
GFGCRGPWDEDDVQCHNHCKSIKGYKGGYCAKGGFVCKCY

SEQ ID NO: 10 - protein, 40 aa, Artificial sequence
  Synthetic construct
GFGCNGPWDEDDKQCHNHCKSIKGYKGGYCAKGGFVCRCY

SEQ ID NO: 11 - protein, 40 aa, Artificial sequence
  Synthetic construct
GFGCNGPWDEDDMRCHNHCKSIKGYRGGYCAKGGFVCKCY

SEQ ID NO: 12 - protein, 40 aa, Artificial sequence
  Synthetic construct
GFGCSGPWSEDDLRCHVHCKSIKGYKGGYCSKGGFVCKCY

SEQ ID NO: 13 - protein, 40 aa, Artificial sequence
  Synthetic construct
GFGCGGPWDEDDLQCHNHCKSIKGYKGGYCAKGGFVCKCY

SEQ ID NO: 14 - protein, 40 aa, Artificial sequence
  Synthetic construct
GFGCGGPWSEDDLQCHQHCKSIKGYKGGYCTKGGFVCKCY

SEQ ID NO: 15 - protein, 40 aa, Artificial sequence
  Synthetic construct
GFGCNGPWNEDDLRCHNHCKSIKGYKGGYCAKGGFVCKCY

SEQ ID NO: 16 - protein, 40 aa, Artificial sequence
  Synthetic construct
GFGCNGPWGEDDMRCHNHCKSIRGYKGGYCAKGGFVCKCY

SEQ ID NO: 17 - protein, 40 aa, Artificial sequence
  Synthetic construct
GFGCNGPWSEDDLRCHNHCKSIKGYRGGYCAKGGFVCKCY

SEQ ID NO: 18 - protein, 40 aa, Artificial sequence
  Synthetic construct
GFGCNGPWSEDDMRCHNHCKSIKGYRGGYCAKGGFVCKCY

SEQ ID NO: 19 - protein, 40 aa, Artificial sequence
  Synthetic construct
GFGCNGPWSEDDMKCHNHCKSIKGYRGGYCAKGGFVCKCY

SEQ ID NO: 20 - protein, 40 aa, Artificial sequence
  Synthetic construct
GFGCNGPWSEDDLKCHNHCKSIKGYKGGYCAKGGFLCKCY

SEQ ID NO: 21 - protein, 40 aa, Artificial sequence
  Synthetic construct
GFGCNGPWSEDDMQCHNHCKSIKGYKGGYCAKGGFVCKCY

SEQ ID NO: 22 - protein, 40 aa, Artificial sequence
  Synthetic construct
GFGCSGPWSEDDMQCHNHCKSIKGYKGGYCAKGGFVCKCY

SEQ ID NO: 23 - protein, 40 aa, Artificial sequence
  Synthetic construct
GFGCNGPWGEDDMQCHNHCKSIKGYKGGYCAKGGFVCKCY

SEQ ID NO: 24 - protein, 40 aa, Artificial sequence
  Synthetic construct
GFGCNGPWDENDMQCHNHCKSIKGYKGGYCAKGGFVCKCY

SEQ ID NO: 25 - protein, 40 aa, Artificial sequence
  Synthetic construct
GFGCSGPWSEDDQQCHNHCKSIKGYKGGYCAKGGFLCKCY

SEQ ID NO: 26 - protein, 40 aa, Artificial sequence
  Synthetic construct
GFGCNGPWSEDDMLCHNHCKSIKGYKGGYCAKGGFVCKCY

SEQ ID NO: 27 - protein, 40 aa, Artificial sequence
  Synthetic construct
GFGCNGPWDEGDMQCHNHCKSIKGYRGGYCAKGGFVCKCY

(19) United States
(12) Patent Application Publication
(10) Pub. No.: US 2009/0227506 A1
(43) Pub. Date: Sep. 10, 2009

(54) USE OF DEFENSINS AGAINST TUBERCULOSIS

(75) Inventors: Hans-Henrik Kristensen Hogenhaug, Sollerod (DK); Gary K. Schoolnik, Palo Alto, CA (US); Sidharth Chopra, Menlo Park, CA (US); Dorotea Raventos Segura, Humlebaek (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/397,796

(22) Filed: Mar. 4, 2009

Related U.S. Application Data
(60) Provisional application No. 61/043,155, filed on Apr. 8, 2008.

(30) Foreign Application Priority Data
Mar. 7, 2008 (EP) 08152499.3

(51) Int. Cl. A61K 38/00; C07K 14/00; C12N 1/20

(52) U.S. Cl. 514/12; 530/350; 435/253.1

(57) ABSTRACT

The present invention relates to a method for killing or inhibiting cells of the genus Mycobacterium, in particular M. tuberculosis, with certain defensins.

### CROSS-REFERENCE TO RELATED APPLICATIONS

This application claims priority or the benefit under 35 U.S.C. 119 of European application no. 08152499.3 filed Mar. 7, 2008 and U.S. provisional application No. 61/043,155 filed Apr. 8, 2008, the contents of which are fully incorporated herein by reference.

### CROSS-REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to tuberculosis treatment, such as treatment of diseases mediated by Mycobacterium, e.g., Mycobacterium tuberculosis, with defensins.

2. Description of the Related Art

Tuberculosis is an infectious disease mediated by infection with Mycobacterium tuberculosis. Tuberculosis is a major disease in developing countries, as well as an increasing problem in developed areas of the world. Although the infection may be asymptomatic for a considerable period of time, the disease is most commonly manifested as an acute inflammation of the lungs, resulting in fever and a nonproductive cough. If untreated, serious complications and death typically result. ‘Tuberculosis may be generally controlled by antibiotic therapy, such as by treatment with Isoniazid, see, e.g., The Merck Index, 12th edition, item 5203; Rifampin (Rifampicin), see, e.g., The Merck Index, 12th edition, item 8382, Streptomycin, see, e.g., The Merck Index, 12th edition, item 8983; but a major problem is the development of strain drug resistance against such antibiotics.

It is an object of the present invention to provide defensin based drugs, and methods of using these, for the treatment of diseases mediated by Mycobacterium, e.g., Mycobacterium tuberculosis.

### SUMMARY OF THE INVENTION

We have now found that certain defensin variants show excellent activity against Mycobacterium tuberculosis, and can be used in the treatment of diseases caused by Mycobacterium, such as tuberculosis.

In one aspect the present invention provides the use of a variant of a parent defensin, comprising a substitution at one or more positions corresponding to positions 5, 9, 11, 13, 14, 17, 20, 23, 26, 31, 36 and 38 of the mature polypeptide of SEQ ID NO: 2, for the manufacturing of a medicament for therapeutic treatment of diseases mediated by Mycobacterium, such as tuberculosis; wherein the variant is capable of killing or inhibiting Mycobacterium tuberculosis cells; and wherein the parent defensin is a polypeptide comprising an amino acid sequence having at least 90% identity to the mature polypeptide of SEQ ID NO: 2, or a polypeptide encoded by a polynucleotide that hybridizes under high stringency conditions with the mature polypeptide coding sequence of SEQ ID NO: 1, or its complementary strand.

In a second aspect, the present invention provides a variant of a parent defensin, comprising a substitution at one or more positions corresponding to positions 5, 9, 11, 13, 14, 17, 20, 23, 26, 31, 36 and 38 of the polypeptide of SEQ ID NO: 2, for therapeutic treatment of diseases mediated by Mycobacterium, such as tuberculosis; wherein the variant is capable of killing or inhibiting Mycobacterium tuberculosis cells; and wherein the parent defensin is a polypeptide comprising an amino acid sequence having at least 90% identity to the mature polypeptide of SEQ ID NO: 2, or a polypeptide encoded by a polynucleotide that hybridizes under high stringency conditions with the mature polypeptide coding sequence of SEQ ID NO: 1, or its complementary strand.

In a third aspect the present invention provides a method for killing or inhibiting Mycobacterium cells, comprising contacting the Mycobacterium cells with a variant of a parent defensin, comprising a substitution at one or more positions corresponding to positions 5, 9, 11, 13, 14, 17, 20, 23, 26, 31, 36 and 38 of the polypeptide of SEQ ID NO: 2; wherein the parent defensin is a polypeptide comprising an amino acid sequence having at least 90% identity to the mature polypeptide of SEQ ID NO: 2, or a polypeptide encoded by a polynucleotide that hybridizes under high stringency conditions with the mature polypeptide coding sequence of SEQ ID NO: 1, or its complementary strand.

In another aspect the present invention provides a method of treating diseases mediated by Mycobacterium, comprising administering to a subject in need of such treatment an effective, e.g., an anti-mycobacterium effective; amount of a variant of a parent defensin, wherein the variant comprises a substitution at one or more positions corresponding to positions 5, 9, 11, 13, 14, 17, 20, 23, 26, 31, 36 and 38 of the polypeptide of SEQ ID NO: 2; and wherein the parent defensin is a polypeptide comprising an amino acid sequence having at least 90% identity to the mature polypeptide of SEQ ID NO: 2, or a polypeptide encoded by a polynucleotide that hybridizes under high stringency conditions with the mature polypeptide coding sequence of SEQ ID NO: 1, or its complementary strand.

Pathogenic Mycobacterium includes Mycobacterium tuberculosis, Mycobacterium bovis, Mycobacterium kansasii, Mycobacterium leprae, Mycobacterium ulcerans, and Mycobacterium avium. Diseases mediated by Mycobacterium include mycobacterium infections. Treatment includes treatment and prophylaxis. A defensin variant for use according to the present invention or for treating diseases according to the present invention is designated hereinafter as “a defensin(s) of (according to) the present invention”.

### DETAILED DESCRIPTION OF THE INVENTION

The present invention relates to pharmaceuticals, and methods of using these for treatment of diseases mediated by Mycobacterium, which include variants of a parent defensin, comprising a substitution at one or more (several) positions corresponding to positions 5, 9, 11, 13, 14, 17, 20, 23, 26, 31, 36 and 38 of the mature polypeptide of SEQ ID NO: 2, wherein the variant is capable of killing or inhibiting growth of Mycobacterium tuberculosis.

### DEFINITIONS

Variant: The term “variant” is defined herein as a polypeptide comprising an alteration, such as a substitution, insertion, and/or deletion, of one or more (several) amino acid residues at one or more (several) specific positions of the mature polypeptide of SEQ ID NO: 2. The altered polynucleotide is obtained through human intervention by modification of the polynucleotide sequence disclosed in SEQ ID NO: 1; or a homologous sequence thereof.

Defensin: The term “defensin” as used herein refers to polypeptides recognized by a person skilled in the art as belonging to the defensin class of antimicrobial peptides. To determine if a polypeptide is a defensin according to the invention, the amino acid sequence is preferably compared with the hidden markov model profiles (HMM profiles) of the PFAM database by using the freely available HMMER software package (see Example 1).

The PFAM defensin families include Defensin_{—}1 or “Mammalian defensin” (accession no. PF00323), Defensin_{—}2 or “Arthropod defensin” (accession no. PF01097), Defensin_beta or “Beta Defensin” (accession no. PF00711), Defensin_propep or “Defensin propeptide” (accession no. PF00879) and Gamma-thionin or “Gamma-thionins family” (accession no. PF00304).

The defensins may belong to the alpha-defensin class, the beta-defensin class, the theta-defensin class, the insect or arthropod defensin classes, or the plant defensin class.

In an embodiment, the amino acid sequence of a defensin according to the invention comprises 4, 5, 6, 7, or 8 cysteine residues, preferably 4, 5, or 6 cysteine residues, more preferably 4 or 6 cysteine residues, and most preferably 6 cysteine residues.

The defensins may also be synthetic defensins sharing the characteristic features of any of the defensin classes.

Examples of such defensins include, but are not limited to, α-Defensin HNP-1 (human neutrophil peptide) HNP-2 and HNP-3; β-Defensin-12, Drosomycin, Heliomicin, γ1-purothionin, Insect defensin A, and the defensins disclosed in PCT applications WO 99/53053, WO 02/06324, WO 02/085934, WO 03/044049, WO 2006/050737 and WO 2006/053565.

Parent Defensin: The term “parent” defensin as used herein means a defensin to which a modification, e.g., substitution(s), insertion(s), deletion(s), and/or truncation(s), is made to produce the defensin variants used in the present invention. This term also refers to the polypeptide with which a variant is compared and aligned. The parent may be a naturally occurring (wild-type) polypeptide or a variant. For instance, the parent polypeptide may be a variant of a naturally occurring polypeptide which has been modified or altered in the amino acid sequence. A parent may also be an allelic variant, which is a polypeptide encoded by any of two or more alternative forms of a gene occupying the same chromosomal locus.

Isolated variant or polypeptide: The term “isolated variant” or “isolated polypeptide” as used herein refers to a variant or a polypeptide that is isolated from a source. In one aspect, the variant or polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure variant or polypeptide: The term “substantially pure variant” or “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure variant or polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The variants and polypeptides of the present invention are preferably in a substantially pure form. This can be accomplished, for example, by preparing the variant or polypeptide by well-known recombinant methods or by classical purification methods.

Mature polypeptide: The term “mature polypeptide” is defined herein as a polypeptide having defensin activity that is in its final form following translation and any post-translational modifications, such as N-terminal processing, C-terminal truncation, glycosylation, phosphorylation, etc. In one aspect, the mature polypeptide is amino acids 1 to 40 of SEQ ID NO: 2 based on the SignalP program that predicts amino acids −55 to −33 of SEQ ID NO: 2 are a signal peptide, and the occurrence of a kex-site at amino acids −2 to −1 of SEQ ID NO: 2.

Mature polypeptide coding sequence: The term “mature polypeptide coding sequence” is defined herein as a nucleotide sequence that encodes a mature polypeptide having defensin activity. In one aspect, the mature polypeptide coding sequence is nucleotides 166 to 285 of SEQ ID NO: 1 based on the SignalP program that predicts nucleotides 1 to 69 of SEQ ID NO: 1 encode a signal peptide, and the occurrence of a kex-site at amino acids −2 to −1 of SEQ ID NO: 1.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment).

### Conventions for Designation of Variants

For purposes of the present invention, the amino acid sequence of the defensin disclosed in SEQ ID NO: 2 is used to determine the corresponding amino acid residue in another defensin. The amino acid sequence of another defensins is aligned with the amino acid sequence of the defensin disclosed in SEQ ID NO: 2, and based on the alignment the amino acid position number corresponding to any amino acid residue in the amino acid sequence of the defensin disclosed in SEQ ID NO: 2 can be determined.

An alignment of polypeptide sequences may be made, for example, using “ClustalW” (Thompson, J. D., Higgins, D. G. and Gibson, T. J., 1994, CLUSTAL W: Improving the sensitivity of progressive multiple sequence alignment through sequence weighting, positions-specific gap penalties and weight matrix choice, Nucleic Acids Research 22: 4673-4680). An alignment of DNA sequences may be done using the polypeptide alignment as a template, replacing the amino acids with the corresponding codon from the DNA sequence.

Pairwise sequence comparison algorithms in common use are adequate to detect similarities between polypeptide sequences that have not diverged beyond the point of approximately 20-30% sequence identity (Doolittle, 1992, Protein Sci. 1: 191-200; Brenner et al., 1998, Proc. Natl. Acad. Sci. USA 95, 6073-6078). However, truly homologous polypeptides with the same fold and similar biological function have often diverged to the point where traditional sequence-based comparison fails to detect their relationship (Lindahl and Elofsson, 2000, J. Mol. Biol. 295: 613-615). Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide of interest has one or more (several) representatives in the protein structure databases. Programs such as GenTHREADER (Jones 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide of interest, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Eng. 11: 739-747), and implementations of these algorithms can additionally be utilized to query structure databases with a structure of interest in order to discover possible structural homologs (e.g., Holm and Park, 2000, Bioinformatics 16: 566-567). These structural alignments can be used to predict the structurally and functionally corresponding amino acid residues in proteins within the same structural superfamily. This information, along with information derived from homology modeling and profile searches, can be used to predict which residues to mutate when moving mutations of interest from one protein to a close or remote homolog.

In describing the various defensin variants of the present invention, the nomenclature described below is adapted for ease of reference. In all cases, the accepted IUPAC single letter or triple letter amino acid abbreviation is employed.

For an amino acid substitution, the following nomenclature is used: Original amino acid, position, substituted amino acid. Accordingly, the substitution of threonine with alanine at position 226 is designated as “Thr226Ala” or “T226A”. Multiple mutations are separated by addition marks (“+”), e.g., “Gly205Arg+Ser411Phe” or “G205R+S411F”, representing mutations at positions 205 and 411 substituting glycine (G) with arginine (R), and serine (S) with phenylalanine (F), respectively.

### Parent Defensin

In the present invention, the parent defensin is (a) a polypeptide comprising an amino acid sequence having at least 90% identity with the mature polypeptide of SEQ ID NO: 2; (b) a polypeptide encoded by a polynucleotide that hybridizes under at least high stringency conditions with the mature polypeptide coding sequence of SEQ ID NO: 1, a complementary strand thereof; or a polypeptide encoded by a polynucleotide comprising a nucleotide sequence having at least 80% identity with the mature polypeptide coding sequence of SEQ ID NO: 1.

In a first aspect, the parent defensins comprise an amino acid sequence having a degree of identity to the mature polypeptide of SEQ ID NO: 2 of at least 80%, preferably at least 85%, more preferably at least 90%, most preferably at least 95%, and even most preferably at least 96%, at least 97%, at least 98%, or at least 99%, which is capable of killing or inhibiting growth of Mycobacterium tuberculosis (hereinafter “homologous polypeptides”). In one aspect, the homologous polypeptides have an amino acid sequence that differs by ten amino acids, preferably by five amino acids, more preferably by four amino acids, even more preferably by three amino acids, most preferably by two amino acids, and even most preferably by one amino acid from the mature polypeptide of SEQ ID NO: 2.

Substantially homologous parent defensins may have one or more (several) amino acid substitutions, deletions and/or insertions. These changes are preferably of a minor nature, that is conservative amino acid substitutions as described above and other substitutions that do not significantly affect the three-dimensional folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a polyhistidine tract, or protein A (Nilsson et al., 1985, EMBO J. 4: 1075; Nilsson et al., 1991, Methods Enzymol. 198: 3. See, also, in general, Ford et al., 1991, Protein Expression and Purification 2: 95-107.

Although the changes described above preferably are of a minor nature, such changes may also be of a substantive nature such as fusion of larger polypeptides of up to 300 amino acids or more both as amino- or carboxyl-terminal extensions.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type defensin. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

The parent defensin preferably comprises the amino acid sequence of SEQ ID NO: 2 or an allelic variant thereof. In one aspect, the parent defensin comprises the amino acid sequence of SEQ ID NO: 2. In another aspect, the parent defensin comprises the mature polypeptide of SEQ ID NO: 2. In another aspect, the parent defensin comprises amino acids 1 to 40 of SEQ ID NO: 2, or an allelic variant thereof. In another aspect, the parent defensin comprises amino acids 1 to 40 of SEQ ID NO: 2. In another aspect, the parent defensin consists of the amino acid sequence of SEQ ID NO: 2 or an allelic variant thereof. In another aspect, the parent defensin consists of the amino acid sequence of SEQ ID NO: 2. In another aspect, the parent defensin consists of the mature polypeptide of SEQ ID NO: 2. In another aspect, the parent defensin consists of amino acids 1 to 40 of SEQ ID NO: 2 or an allelic variant thereof. In another aspect, the parent defensin consists of amino acids 1 to 40 of SEQ ID NO: 2.

In a second aspect, the parent defensins are encoded by polynucleotides that hybridize under medium stringency conditions, preferably medium-high stringency conditions, more preferably high stringency conditions, and most preferably very high stringency conditions with (i) the mature polypeptide coding sequence of SEQ ID NO: 1, or a subsequence thereof (J. Sambrook, E. F. Fritsch, and T. Maniatis, 1989, Molecular Cloning, A Laboratory Manual, 2d edition, Cold Spring Harbor, N.Y.). In one aspect, the complementary strand is the full-length complementary strand of the mature polypeptide coding sequence of SEQ ID NO: 1.

For long polynucleotides of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long polynucleotides of at least 100 nucleotides in length, the carrier material is finally washed three times each for 15 minutes using 2×SSC, 0.2% SDS preferably at 45° C. (very low stringency), more preferably at 50° C. (low stringency), more preferably at 55° C. (medium stringency), more preferably at 60° C. (medium-high stringency), even more preferably at 65° C. (high stringency), and most preferably at 70° C. (very high stringency).

For short polynucleotides that are about 15 nucleotides to about 70 nucleotides in length, stringency conditions are defined as prehybridization, hybridization, and washing post hybridization at about 5° C. to about 10° C. below the calculated Tₘ using the calculation according to Bolton and McCarthy (1962, Proceedings of the National Academy of Sciences USA 48: 1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally.

For short polynucleotides that are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

In a third aspect, the parent defensin is encoded by a polynucleotide comprising or consisting of a nucleotide sequence having a degree of identity to the mature polypeptide coding sequence of SEQ ID NO: 1 of preferably at least 80%, preferably at least 85%, more preferably at least 90%, most preferably at least 95%, and even most preferably 96%, 97%, 98%, or 99%, which encode an active polypeptide. In one aspect, the mature polypeptide coding sequence is nucleotides 166 to 285 of SEQ ID NO: 1.

The parent defensin may be obtained from microorganisms of any genus. For purposes of the present invention, the term “obtained from” as used herein in connection with a given source shall mean that the parent defensin encoded by a polynucleotide is produced by the source or by a cell in which the polynucleotide from the source has been inserted. In one aspect, the parent defensin is secreted extracellularly.

The parent defensin may be a fungal defensin. In another aspect, the fungal defensin is a yeast defensin such as a Candida, Kluyveromyces, Pichia, Saccharomyces, Schizosaccharomyces, or Yarrowia defensin. In another aspect, the fungal defensin is a filamentous fungal defensin such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria defensin.

In another aspect, the parent defensin is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis defensin.

In another aspect, the parent defensin is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, or Trichoderma viride defensin.

In another aspect, the parent defensin is a Pseudoplectania nigrella defensin, and most preferably, the Pseudoplectania nigrella defensin of SEQ ID NO: 2 or the mature polypeptide thereof.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

The parent defensin may also be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. The polynucleotide encoding a defensin may then be derived by similarly screening a genomic or cDNA library of another microorganism or mixed DNA sample. Once a polynucleotide encoding a defensin has been detected with suitable probe(s) as described herein, the sequence may be isolated or cloned by utilizing techniques that are known to those of ordinary skill in the art (see, e.g., J. Sambrook, E. F. Fritsch, and T. Maniatus, 1989, Molecular Cloning, A Laboratory Manual, 2d edition, Cold Spring Harbor, N.Y.). As defined herein, an “isolated” defensin is a polypeptide that is essentially free of other non-defensin polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

The parent defensin can also include fused polypeptides or cleavable fusion polypeptides in which another polypeptide is fused at the N-terminus or the C-terminus of the polypeptide or fragment thereof. A fused polypeptide is produced by fusing a polynucleotide (or a portion thereof) encoding another polypeptide to a polynucleotide (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

### Preparation of Variants

Variants of a parent defensin can be prepared according to any mutagenesis procedure known in the art, such as site-directed mutagenesis, synthetic gene construction, semi-synthetic gene construction, random mutagenesis, shuffling, etc.

Site-directed mutagenesis is a technique in which one or several mutations are created at a defined site in a polynucleotide molecule encoding the parent defensin. The technique can be performed in vitro or in vivo.

Synthetic gene construction entails in vitro synthesis of a designed polynucleotide molecule to encode a polypeptide molecule of interest. Gene synthesis can be performed utilizing a number of techniques, such as the multiplex microchip-based technology described by Tian et al. (Tian et. al., Nature 432: 1050-1054) and similar technologies wherein olgionucleotides are synthesized and assembled upon photo-programmable microfluidic chips.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent defensin and subsequent ligation of an oligonucleotide containing the mutation in the polynucleotide. Usually the restriction enzyme that digests at the plasmid and the oligonucleotide is the same, permitting sticky ends of the plasmid and insert to ligate to one another. See, for example, Scherer and Davis, 1979, Proc. Natl. Acad. Sci. USA 76: 4949-4955; and Barton et al., 1990, Nucleic Acids Research 18: 7349-4966.

Site-directed mutagenesis can be accomplished in vivo by methods known in the art. See, for example, U.S. Patent Application Publication 2004/0171154; Storici et al., 2001, Nature Biotechnology 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants of a parent defensin.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204) and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide fragments may then be shuffled.

### Variants

In the present invention, the isolated variants of a parent defensin comprise a substitution at one or more (several) positions corresponding to positions 5, 9, 11, 13, 14, 17, 20, 23, 26, 31, 36, and 38, wherein the variant, which is capable of killing or inhibiting growth of Mycobacterium tuberculosis, comprises an amino acid sequence having a degree of identity of at least 80%, preferably at least 85%, more preferably at least 90%, most preferably at least 95%, and even most preferably at least about 97% to the amino acid sequence of the parent defensin.

In one aspect, the number of amino acid substitutions in the variants of the present invention comprises preferably 4 substitutions, more preferably 3 substitutions, even more preferably 2 substitutions, and most preferably 1 substitution. In another aspect, the number of amino acid substitutions in the variants of the present invention consists of preferably 4 substitutions, more preferably 3 substitutions, even more preferably 2, and most preferably 1 substitution.

In one aspect, a variant of a parent defensin comprises a substitution at one or more (several) positions corresponding to positions 5, 9, 11, 13, 14, 17, 20, 23, 26, 31, 36, and 38. In another aspect, a variant of a parent defensin comprises substitutions at two or more positions corresponding to positions 5, 9, 11, 13, 14, 17, 20, 23, 26, 31, 36, and 38. In another aspect, a variant of a parent defensin comprises substitutions at three or more positions corresponding to positions 5, 9, 11, 13, 14, 17, 20, 23, 26, 31, 36, and 38. In another aspect, a variant of a parent defensin comprises substitutions at positions corresponding to positions 5, 9, 11, 13, 14, 17, 20, 23, 26, 31, 36, and 38.

In one aspect, the variant comprises a substitution at a position corresponding to position 5. In another aspect, the variant comprises a substitution at a position corresponding to position 5 with Arg, Gly, or Ser. In another aspect, the variant comprises the substitution N5R, N5G or N5S of the mature polypeptide of SEQ ID NO: 2.

In another aspect, the variant comprises a substitution at a position corresponding to position 9. In another aspect, the variant comprises a substitution at a position corresponding to position 9 with Asn, Gly, or Ser. In another aspect, the variant comprises the substitution D9N, D9G or D9S of the mature polypeptide of SEQ ID NO: 2.

In another aspect, the variant comprises a substitution at a position corresponding to position 11. In another aspect, the variant comprises a substitution at a position corresponding to position 11 with Asn or Gly. In another aspect, the variant comprises the substitution D11N or D11G of the mature polypeptide of SEQ ID NO: 2.

In another aspect, the variant comprises a substitution at a position corresponding to position 13. In another aspect, the variant comprises a substitution at a position corresponding to position 13 with Leu, Lys, or Val. In another aspect, the variant comprises the substitution M13L, M13K or M13V of the mature polypeptide of SEQ ID NO: 2.

In another aspect, the variant comprises a substitution at a position corresponding to position 14. In another aspect, the variant comprises a substitution at a position corresponding to position 14 with Arg, Leu, Lys, or Phe. In another aspect, the variant comprises the substitution Q14F, Q14L, Q14K or Q14R of the mature polypeptide of SEQ ID NO: 2.

In another aspect, the variant comprises substitutions at positions corresponding to positions selected from the group consisting of (a) positions 5 and 9; positions 5 and 13; positions 5 and 14; positions 9 and 13; positions 9 and 14; positions 13 and 14; positions 11 and 5; positions 11 and 9; positions 11 and 13; or positions 11 and 14 of the mature polypeptide of SEQ ID NO: 2; (b) positions 5, 9, and 13; positions 5, 13, and 14; positions 9, 13, and 14; or positions 5, 9, and 14 of the mature polypeptide of SEQ ID NO: 2; and (c) positions 5, 9, 13, and 14; or positions 5, 9, 11, 13, and 14 of the mature polypeptide of SEQ ID NO: 2.

In another aspect, the variant comprises a substitution at a position corresponding to

position 5 is Gly, Ser or Arg;
position 9 is Gly, Ser or Asn;
position 11 is Asn or Gly;
position 13 is Leu, Val or Lys;
position 14 is Leu, Phe, Lys or Arg;
position 17 is Val or Gln;
position 20 is Arg;
position 23 is Arg;
position 26 is Arg;
position 31 is Ser or Thr;
position 36 is Leu; and
position 38 is Arg.

In another aspect, the variant comprises one or more substitutions selected from the group consisting of:

### N5G, N5S or N5R;

### D9G, D9S or D9N;

### D11N or D11G;

### M13L, M13V or M13K;

### Q14L, Q14F, Q14K or Q14R;

### N17V or N17Q;

### K20R;

### K23R;

### K26R;

### A31S or A31T;

### V36L; and

### K38R.

In another aspect, the variant comprises an amino acid sequence having at least 80% identity, preferably at least 85% identity, more preferably at least 90% identity, and most preferably at least 95% identity to the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

In another aspect, the variant comprises or consists of the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

Other Polypeptides Capable of Killing or Inhibiting Mycobacterium tuberculosis

The present invention also relates to isolated polypeptides capable of killing or inhibiting growth of Mycobacterium tuberculosis, wherein the amino acid sequences of the polypeptides differ from SEQ ID NO: 2 at one or more (several) positions corresponding to positions 5, 9, 11, 13, 14, 17, 20, 23, 26, 31, 36, and 38 of SEQ ID NO: 2.

In one aspect, the amino acid sequence of the polypeptide differs from the mature polypeptide of SEQ ID NO: 2 by preferably 4 amino acids, more preferably 3 amino acids, even more preferably 2 amino acids, and most preferably 1 amino acid.

In one aspect, the amino acid sequence of the polypeptide differs from SEQ ID NO: 2 at one or more (several) positions corresponding to positions 5, 9, 11, 13, 14, 17, 20, 23, 26, 31, 36, and 38. In another aspect, the amino acid sequence of the polypeptide differs from SEQ ID NO: 2 at two or more positions corresponding to positions 5, 9, 11, 13, 14, 17, 20, 23, 26, 31, 36, and 38. In another aspect, the amino acid sequence of the polypeptide differs from SEQ ID NO: 2 at three or more positions corresponding to positions 5, 9, 11, 13, 14, 17, 20, 23, 26, 31, 36, and 38. In another aspect, the amino acid sequence of the polypeptide differs from SEQ ID NO: 2 at positions corresponding to positions 5, 9, 11, 13, 14, 17, 20, 23, 26, 31, 36, and 38.

In one aspect, the amino acid sequence of the polypeptide differs from SEQ ID NO: 2 at a position corresponding to position 5. In another aspect, the amino acid sequence of the polypeptide differs from SEQ ID NO: 2 at a position corresponding to position 5 by Arg, Gly, or Ser. In another aspect, the amino acid sequence of the polypeptide differs from SEQ ID NO: 2 by Arg, Gly, or Ser at position 5 of the mature polypeptide of SEQ ID NO: 2.

In another aspect, the amino acid sequence of the polypeptide differs from SEQ ID NO: 2 at a position corresponding to position 9. In another aspect, the amino acid sequence of the polypeptide differs from SEQ ID NO: 2 at a position corresponding to position 9 by Gly, Ser, or Asn. In another aspect, the amino acid sequence of the polypeptide differs from SEQ ID NO: 2 by Gly, Ser, or Asn at position 9 of the mature polypeptide of SEQ ID NO: 2.

In another aspect, the amino acid sequence of the polypeptide differs from SEQ ID NO: 2 at a position corresponding to position 11. In another aspect, the amino acid sequence of the polypeptide differs from SEQ ID NO: 2 at a position corresponding to position 11 by Asn or Gly. In another aspect, the amino acid sequence of the polypeptide differs from SEQ ID NO: 2 by Asn or Gly at position 11 of the mature polypeptide of SEQ ID NO: 2.

In another aspect, the amino acid sequence of the polypeptide differs from SEQ ID NO: 2 at a position corresponding to position 13. In another aspect, the amino acid sequence of the polypeptide differs from SEQ ID NO: 2 at a position corresponding to position 13 by Leu, Lys, or Val. In another aspect, the amino acid sequence of the polypeptide differs from SEQ ID NO: 2 by Leu, Lys, or Val at position 13 of the mature polypeptide of SEQ ID NO: 2.

In another aspect, the amino acid sequence of the polypeptide differs from SEQ ID NO: 2 at a position corresponding to position 14. In another aspect, the amino acid sequence of the polypeptide differs from SEQ ID NO: 2 at a position corresponding to position 14 by Phe, Leu, Lys, or Arg. In another aspect, the amino acid sequence of the polypeptide differs from SEQ ID NO: 2 by Phe, Leu, Lys, or Arg at position 14 of the mature polypeptide of SEQ ID NO: 2.

In another aspect, the difference corresponding to

position 5 is Gly, Ser or Arg;
position 9 is Gly, Ser or Asn;
position 11 is Asn or Gly;
position 13 is Leu, Val or Lys;
position 14 is Leu, Phe, Lys or Arg;
position 17 is Val or Gln;
position 20 is Arg;
position 23 is Arg;
position 26 is Arg;
position 31 is Ser or Thr;
position 36 is Leu; and
position 38 is Arg.

In another aspect, the amino acid sequence of the polypeptide differs from SEQ ID NO: 2 at positions corresponding to positions selected from the group consisting of (a) positions 5 and 9; positions 5 and 13; positions 5 and 14; positions 9 and 13; positions 9 and 14; positions 13 and 14; positions 11 and 5; positions 11 and 9; positions 11 and 13; or positions 11 and 14 of the mature polypeptide of SEQ ID NO: 2; (b) positions 5, 9, and 13; positions 5, 13, and 14; positions 9, 13, and 14; or positions 5, 9, and 14 of the mature polypeptide of SEQ ID NO: 2; and (c) positions 5, 9, 13, and 14; or positions 5, 9, 11, 13, and 14 of the mature polypeptide of SEQ ID NO: 2.

### Methods and Uses

The present invention is also directed to methods for using the defensin variants.

The invention relates to the use of a defensin variant of the invention for treating tuberculosis. Further, an antimicrobial polypeptide or composition of the invention may also be used for the manufacture of a medicament for treating tuberculosis.

The defensin variants of the invention may be used as an antimicrobial veterinarian or human therapeutic or prophylactic agent. Thus, defensin variants of the invention may be used in the preparation of veterinarian or human therapeutic agents or prophylactic agents for the treatment of tuberculosis.

The defensin variants of the invention are used in an amount sufficient to kill or inhibit growth of Mycobacterium cells, preferably Mycobacterium tuberculosis.

Formulations of the defensin variants of the invention are administered to a host suffering from or predisposed to a Mycobacterium infection, such as tuberculosis.

Administration may be localized or systemic. Generally the dose of the antimicrobial polypeptides of the invention will be sufficient to decrease the microbial population by at least about 50%, usually by at least 1 log, and may be by 2 or more logs of killing. The compounds of the present invention are administered at a dosage that reduces the microbial population while minimizing any side-effects. It is contemplated that the composition will be obtained and used under the guidance of a physician for in vivo use.

Various methods for administration may be employed. The polypeptide formulation may be given orally, or may be injected intravascularly, subcutaneously, peritoneally, by aerosol, opthalmically, intra-bladder, topically, etc. For example, methods of administration by inhalation are well-known in the art. The dosage of the therapeutic formulation will vary widely, depending on the specific antimicrobial polypeptide to be administered, the nature of the disease, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered once or several times daily, semi-weekly, etc. to maintain an effective dosage level. In many cases, oral administration will require a higher dose than if administered intravenously. The amide bonds, as well as the amino and carboxy termini, may be modified for greater stability on oral administration. For example, the carboxy terminus may be amidated.

### Formulations

The compounds of this invention can be incorporated into a variety of formulations for therapeutic administration. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols. As such, administration of the compounds can be achieved in various ways, including oral, buccal, rectal, parenteral, intraperitoneal, intradermal, transdermal, intracheal, etc., administration. The antimicrobial polypeptides of the invention may be systemic after administration or may be localized by the use of an implant or other formulation that acts to retain the active dose at the site of implantation.

The compounds of the present invention can be administered alone, in combination with each other, or they can be used in combination with other known compounds (e.g., perforin, anti-inflammatory agents, antibiotics, etc.). In pharmaceutical dosage forms, the compounds may be administered in the form of their pharmaceutically acceptable salts. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing the antimicrobial polypeptides of the invention is placed in proximity to the site of infection, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the unit dosage forms of the present invention depend on the particular compound employed and the effect to be achieved, and the pharmacodynamics associated with the compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 pg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will be normally be neutral or acidic lipids, such as cholesterol, phosphatidyl serine, phosphatidyl glycerol, and the like.

For preparing the liposomes, the procedure described by Kato et al., 1991, J. Biol. Chem. 266: 3361 may be used. Briefly, the lipids and lumen composition containing peptides are combined in an appropriate aqueous medium, conveniently a saline medium where the total solids will be in the range of about 1-10 weight percent. After intense agitation for short periods of time, from about 5-60 sec., the tube is placed in a warm water bath, from about 25-40° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

Formulations with Other Active Agents

For use in the subject methods, the antimicrobial polypeptides of the invention may be formulated with other pharmaceutically active agents, particularly other antimicrobial agents. Other agents of interest include a wide variety of antibiotics, as known in the art. Classes of antibiotics include penicillins, e.g., penicillin G, penicillin V, methicillin, oxacillin, carbenicillin, nafcillin, ampicillin, etc.; penicillins in combination with beta-lactamase inhibitors, cephalosporins, e.g., cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g., amphotericin B, nystatin; 5-flucosyn; and azoles, e.g., miconazol, ketoconazol, itraconazol and fluconazol. Antituberculotic drugs include isoniazid, ethambutol, streptomycin and rifampin. Cytokines may also be included in a formulation of the antimicrobial polypeptides of the invention, e.g., interferon gamma, tumor necrosis factor alpha, interleukin 12, etc.

### In Vitro Synthesis

The polypeptides of the invention may be prepared by in vitro synthesis, using conventional methods as known in the art. Various commercial synthetic apparatuses are available, for example automated synthesizers by Applied Biosystems Inc., Beckman, etc. By using synthesizers, naturally occurring amino acids may be substituted with unnatural amino acids, particularly D-isomers (or D-forms) e.g., D-alanine and D-isoleucine, diastereoisomers, side chains having different lengths or functionalities, and the like. The particular sequence and the manner of preparation will be determined by convenience, economics, purity required, and the like.

Chemical linking may be provided to various peptides or proteins comprising convenient functionalities for bonding, such as amino groups for amide or substituted amine formation, e.g., reductive amination, thiol groups for thioether or disulfide formation, carboxyl groups for amide formation, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein

The present invention is further described by the following examples that should not be construed as limiting the scope of the invention.

### EXAMPLES

### Example 1

### Using the HMM Files from the PFAM Database to Identify a Defensin

Sequence analysis using hidden markov model profiles (HMM profiles) may be carried out either online on the Internet or locally on a computer using the well-known HMMER freely available software package. The current version is HMMER 2.3.2 from October 2003.

The HMM profiles may be obtained from the well-known PFAM database. The current version is PFAM 16.0 from November 2004. Both HMMER and PFAM are available for all computer platforms from e.g., Washington University in St. Louis (USA), School of Medicine (pfam.wustl.edu and hmmer.wustl.edu).

If a query amino acid sequence or a fragment thereof belongs to one of the following five PFAM families, the amino acid sequence is a defensin according to the present invention:

Defensin_beta or “Beta Defensin”, accession number: PF00711;
Defensin_propep or “Defensin propeptide”, accession number: PF00879;
Defensin_{—}1 or “Mammalian defensin”, accession number: PF00323;
Defensin_{—}2 or “Arthropod defensin”, accession number: PF01097;
Gamma-thionin or “Gamma-thionins family”, accession number: PF00304.

An amino acid sequence belongs to a PFAM family, according to the present invention, if it generates an E-value which is greater than 0.1, and a score which is larger or equal to zero, when the PFAM database is used online, or when the hmmpfam program (from the HMMER software package) is used locally.

When the sequence analysis is carried out locally using the hmmpfam program, it is necessary to obtain (download) the HMM profiles from the PFAM database. Two profiles exist for each family; xxx_ls.hmm for glocal searches, and xxx_fs.hmm for local searches (“xxx” is the name of the family). That makes a total of ten profiles for the five families mentioned above.

These ten profiles may be used individually, or joined (appended) into a single profile (using a text editor—the profiles are ASCII files) that could be named e.g., defensin.hmm. A query amino acid sequence can then be evaluated by using the following command line:
hmmpfam-E 0.1 defensin.hmm sequence_file
   wherein “sequence_file” is a file with the query amino acid sequence in any of the formats recognized by the HMMER software package.

If the score is larger or equal to zero (0.0), and the E-value is greater than 0.1, the query amino acid sequence is a defensin according to the present invention.

The PFAM database is further described in Bateman et al., 2004, “The Pfam Protein Families Database”, Nucleic Acids Research, Vol. 32 (Database Issue) pp. D138-D141.

### Example 2

### Luciferase-Based Assay for Antimicrobial Activity

Routinely, antimicrobial activity of antibiotics is measured using standard protocols. The potencies are most often expressed as Minimal Inhibitory Concentrations (MICs). To determine the MICs of pathogenic, slow-growing mycobacteria such as M. tuberculosis, several modified systems are available which takes advantage of either radioactivity (BACTEC) or fluorescence (MGIT) as a quantifiable readout. However as these methods both require special equipment, an MIC protocol using a bacterial luciferase was established. Luciferase, once the encoding gene has been transformed into and expressed in a given organism, it can be used as an indicator for the viability of that organism. The use of luciferase (LUX) assay circumvents issues such as slow growth (˜30 days to form colonies on a nutrient plate) and clumping which plague most of the CFU based assays for M. tuberculosis. The results are fast (within 2-4 days) and can give an idea about the potency of a given compound—especially when it is compared to other compounds using the same setup.

In this example, M. tuberculosis H37Rv was transformed with a luciferease-expressing plasmid.

1. With a single glycerol stock of M. tuberculosis, inoculate 50-100 ml of 7H9 (Fisher, Catalog#: 271310) ADC (Fisher, Catalog #: L12240)+0.05% Tween (Sigma, Catalog#: T 8761) in a 1 litre roller bottle. Tighten the cap and incubate the roller bottle at 30-60 rpm in 37° C.
2. Incubate the bottle until OD600 is between 0.5-0.8. This typically takes around 4-7 days.
3. On the day of the experiment, dilute the culture in the morning (6-8 h previously) to OD600˜0.075. Make up the volume to ˜50-100 ml with fresh media and incubate for 6-8 h, such that the OD600 is between 0.125-0.200. This is the experimental culture.
4. Make the 96 well plates with different concentrations of the peptides, keeping in mind that the total volume per well should not exceed 250 microliters.
5. Incubate the plate by sealing it in a gas permeable pouch for 96 h at 37° C.±5% CO₂
6. Remove the plate from the incubator and discard the pouch. Incubate the plate in the hood with its lid open for 60 min, so that it equilibrates to the room temperature.
7. Using the automatic injector of the Luminometer start the luciferase-reaction by injection 25 microliters decanal (1% in 95% Ethanol), read the plate in the luminometer and analyze the data.

A MIC is determined as the concentration of compound that reduces the relative light units (RLU) by 90% (1 log). The MIC of plectasin (SEQ ID NO: 2) was determined to be around 25 micrograms/ml whereas the MIC of the plectasin variant peptide SEQ ID NO: 14 was determined to be around 6 micrograms/ml.

### Example 3

### Validation of the RLU Assay by Confirmation with CFU or Traditional Plate Assay

The validation of the Relative Light Unit assay (RLU) was carried out by comparing it to conventional Colony Forming Unit (CFU) assay. In this assay, the cells were exposed to the different concentrations of the peptide for 96 h and then plated onto 7H10 plates. The plates were incubated at 37° C. for 30 days and the colonies were enumerated.

The MIC of 6.25 micrograms/ml obtained by RLU is equivalent to the MBC obtained by CFU, thus pointing to the fact that the SEQ ID NO: 14 peptide is bactericidal, as is plectasin, against other Gram-positive bacteria.

The conclusion is that the current Luciferase setup can be utilized to accurately determine MIC and that it has potential to be implemented as a high throughput screen.

### Example 4

### Determination of MIC Based on OD₆₀₀

The inhibitory effect of the SEQ ID NO: 14 peptide was also correlated to growth by measuring its effect by OD₆₀₀. NZ2109 at 6.25 micrograms/ml was able to completely inhibit the growth of M. tuberculosis in T-25 flasks.

Taken together, both of these assays in Examples 3 and 4 confirm the MIC (=MBC) at 6.25 micrograms/ml for the SEQ ID NO: 14 peptide.

### Example 5

### Identification of Antimicrobial Peptides with Potent Activity Against Mycobacterium tuberculosis

A number of antimicrobial peptides were tested for their antimicrobial activity against M. tuberculosis H37Rv using the luciferase assay as described in Example 2. The concentrations of the peptides in this specific assay were 25 micrograms/ml. The most potent of these peptides were either plectasin or derivatives containing specific amino acid changes. The peptides, their corresponding amino acid sequence and their degree of inhibition and listed in Table 1 below.

TABLE 1

| | Amino acid substitutions | | Fold of |
|---|---|---|---|
| SEQ ID NO: | compared to SEQ ID NO: 2 | RLU | reduction |
| Buffer | NA | 53503 | 1 |
| control | | | |
| 2 | None | 2070 | 25.7 |
| 3 | Q14K + K26R | 2310 | 23.2 |
| 4 | K26R | 1903 | 28.1 |
| 5 | Q14F | 1567 | 34.1 |
| 6 | Q14R + K26R + K38R | 4483 | 11.9 |
| 7 | Q14R + K20R | 1339 | 40.0 |
| 8 | Q14L | 2145 | 24.9 |
| 9 | N5R + M13V | 1685 | 31.8 |
| 10 | M13K + K38R | 1152 | 46.4 |
| 11 | Q14R + K26R | 3604 | 14.8 |
| 12 | N5S + D9S + M13L + | 3901 | 13.7 |
| | Q14R + N17V + A31S | | |
| 13 | N5G + M13L | 2576 | 20.8 |
| 14 | N5G + D9S + M13L + | 1083 | 49.4 |
| | N17Q + A31T | | |
| 15 | D9N + M13L + Q14R | 4165 | 12.9 |
| 16 | D9G + Q14R + K23R | 4495 | 11.9 |

In a similar experimental setup, other variants of plectasin were tested for their antimicrobial activity against M. tuberculosis H37Rv using the luciferase assay as described in Example 2. The concentrations of the peptides in this specific assay were 6.25 micrograms/ml. The best peptides, all derivatives of plectasin, are listed in Table 2 below.

TABLE 2

| | Amino acid substitutions | | Fold of |
|---|---|---|---|
| SEQ ID NO: | compared to SEQ ID NO: 2 | RLU | reduction |
| Buffer | NA | 75380 | 1 |
| control | | | |
| 17 | D9S + M13L + Q14R + K26R | 8321 | 9.1 |
| 18 | D9S + Q14R + K26R | 7702 | 9.8 |
| 19 | D9S + Q14K + K26R | 7630 | 9.9 |
| 20 | D9S + M13L + Q14K + V36L | 5355 | 14.1 |

All of the peptides above had a MIC of 25 micrograms/ml or lower. A few of the peptides, SEQ ID NO: 14, SEQ ID NO: 7, SEQ ID NO: 9 and SEQ ID NO: 20, were also tested at a lower concentration and had a MIC of 6.25 micrograms/ml. The peptides SEQ ID NO: 17, SEQ ID NO: 18 and SEQ ID NO: 19 almost exhibited the required 10-fold reduction in RLU and hence have MICs very close to 6.25 micrograms/ml.

### Example 6

### Further Antimicrobial Peptides with Potent Activity Against Mycobacterium tuberculosis

Essentially following the procedures outlined in Examples 3 and 4, and as described in the NCCLS guidelines (M24-A), more variants of plectasin were evaluated as shown in Table 3 below. The corresponding MIC values are shown in Table 3.

TABLE 3

| | Amino acid substitutions compared | MIC |
|---|---|---|
| SEQ ID NO: | to SEQ ID NO: 2 | (micrograms/ml) |
| 21 | D9S | 1.5 |
| 22 | N5S + D9S | 3.2 |
| 23 | D9G | 6.2 |
| 24 | D11N | 6.2 |
| 25 | N5S + D9S + M13Q + V36L | 6.2 |
| 26 | D9S + Q14L | 6.2 |
| 27 | D11G + K26R | 6.2 |

The results shown in Table 3 indicate that all the tested peptides exhibit potent activity against Mycobacterium tuberculosis.

### Example 7

### The SEQ ID NO: 14 Peptide is Active Against Early Stationary Cells

H37Rv expressing LUX was tested simultaneously at ˜0.1 OD₆₀₀ and ˜1.0 OD₆₀₀ in a 96 well plate with 6.25 micrograms/ml of the SEQ ID NO: 14 peptide. The plates were sealed and incubated at 37° C. for 96 h with 5% CO₂. Afterwards, the light units were read. An OD₆₀₀ of ˜1.0 in contrast to later growth stages was chosen because at OD₆₀₀'s higher than ˜1, the correlation between LUX and OD seems to get off the curve. Also, later growth stages make the experiments more complicated because of too much visible clumping that would interfere with any microbiological procedure.

As can be seen from the data, the SEQ ID NO: 14 peptide does not seem to significantly differentiate between physiology of the bacteria tested i.e., approx. 0.1 OD₆₀₀ and approx. 1.0 OD₆₀₀. Thus, the SEQ ID NO: 14 peptide express potent activity against organisms in both early log and early stationary phases of growth.

1. A variant of a parent defensin, comprising a substitution at one or more positions corresponding to positions 5, 9, 11, 13, 14, 17, 20, 23, 26, 31, 36 and 38 of the polypeptide of SEQ ID NO: 2, wherein the variant is capable of killing or inhibiting Mycobacterium tuberculosis cells; and wherein the parent defensin is a polypeptide comprising an amino acid sequence having at least 90% identity to the mature polypeptide of SEQ ID NO: 2, or a polypeptide encoded by a polynucleotide that hybridizes under high stringency conditions with the mature polypeptide coding sequence of SEQ ID NO: 1, or its complementary strand.

2. The variant of claim 1, wherein the parent defensin is a polypeptide comprising an amino acid sequence having at least 95% identity to the mature polypeptide of SEQ ID NO: 2, preferably wherein the parent defensin comprises or consists of the mature polypeptide of SEQ ID NO: 2.

3. The variant of claim 1, wherein the parent defensin comprises or consists of the mature polypeptide of SEQ ID NO: 2.

4. The variant of claim 1, wherein the substitution at a position corresponding to
position 5 is Gly, Ser or Arg;
position 9 is Gly, Ser or Asn;
position 11 is Asn or Gly;
position 13 is Leu, Val or Lys;
position 14 is Leu, Phe, Lys or Arg;
position 17 is Val or Gln;
position 20 is Arg;
position 23 is Arg;
position 26 is Arg;
position 31 is Ser or Thr;
position 36 is Leu; and
position 38 is Arg.

5. The variant of claim 1, which comprises substitutions at positions corresponding to positions selected from the group consisting of (a) positions 5 and 9; positions 5 and 13; positions 5 and 14; positions 9 and 13; positions 9 and 14; positions 13 and 14; positions 11 and 5; positions 11 and 9; positions 11 and 13; or positions 11 and 14 of the mature polypeptide of SEQ ID NO: 2; (b) positions 5, 9, and 13; positions 5, 13, and 14; positions 9, 13, and 14; or positions 5, 9, and 14 of the mature polypeptide of SEQ ID NO: 2; and (c) positions 5, 9, 13, and 14; or positions 5, 9, 11, 13, and 14 of the mature polypeptide of SEQ ID NO: 2.

6. The variant of claim 1, wherein the variant comprises one or more substitutions selected from the group consisting of:
N5G, N5S or N5R;
D9G, D9S or D9N;
D11N or D11G;
M13L, M13V or M13K;
Q14L, Q14F, Q14K or Q14R;
N17V or N17Q;
K20R;
K23R;
K26R;
A31S or A31T;
V36L; and
K38R.

7. The variant of claim 1, wherein the variant comprises an amino acid sequence having at least 90% identity to the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

8. The variant of claim 1, wherein the variant comprises an amino acid sequence having at least 95% identity to the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

9. The variant of claim 1, wherein the variant comprises or consists of the amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

10. A method for killing or inhibiting Mycobacterium cells, comprising contacting the Mycobacterium cells with a variant of claim 1.

11. A method of treating diseases mediated by Mycobacterium, comprising administering to a subject in need of such treatment an effective amount of a variant of claim 1.

12. A polypeptide capable of killing or inhibiting Mycobacterium tuberculosis cells for therapeutic treatment of tuberculosis; wherein the amino acid sequence of the polypeptide differs from SEQ ID NO: 2 at one or more positions corresponding to positions 5, 9, 11, 13, 14, 17, 20, 23, 26, 31, 36 and 38 of the mature polypeptide of SEQ ID NO: 2.

13. A method for killing or inhibiting Mycobacterium cells, comprising contacting the Mycobacterium cells with a polypeptide of claim 12.

14. A method of treating diseases mediated by Mycobacterium, comprising administering to a subject in need of such treatment an effective amount of a polypeptide of claim 12.
